(19) United States
(12) Patent Application Publication
(10) Pub. No.: US 2003/0198690 A1
(43) Pub. Date: Oct. 23, 2003

(54) Device and method for treating restenosis

(75) Inventors: Jacob George, Tel Aviv (IL); Gad Keren, Kiryat Ono (IL)

Correspondence Address: BROWDY AND NEIMARK, P.L.L.C., 624 NINTH STREET, NW, SUITE 300, WASHINGTON, DC, 20001-5303, US

(73) Assignee: Medical Research Fund of Tel Aviv Sourasky Medical Center, Tel Aviv (IL)

(21) Appl. No.: 10/126,979

(22) Filed: Apr. 22, 2002

(51) Int. Cl. A61K 33/32; A61M 31/00

(52) U.S. Cl. 424/641; 604/500

(57) ABSTRACT

A method of reducing neointiminal proliferation in a blood vessel, in particular as a result of restenosis, comprising administrating zinc to the blood vessel. Also disclosed is a stent comprising zinc for use in the method.

### FIELD OF THE INVENTION

This invention relates to a method of preventing and/or treating restenosis, and to devices and compositions which may be used in the method.

### BACKGROUND OF THE INVENTION

Restenosis has been defined as the arterial healing response after injury incurred during transluminal coronary revascularization (Lowe, H. C. et al. (2002) Coronary in-stent restenosis: current status and future strategies J. Amer. Coll. Cardiol. 39(2):183-193).

Attempts at prevention of in-stent restenosis include improved stent implantation, improved stent design, radiation therapy, and drug delivery. Post-restenosis treatment is generally carried out by balloon angioplasty and directional atherectomy. However, the success rate in all of the above strategies is unacceptably low. None of the available interventional modalities provides optimal acute results, and long-term results are even poorer (Hoffman, R. and Mintz, G. S. (2000) Coronary in-stent restenosis—predictors, treatment and prevention Europ. Heart J. 21:1739-1749). The magnitude of the restenosis phenomenon in interventional cardiology is such that, due to a lack of appropriate methods of treatment, many patients are sent to surgery rather than being treated by a second angioplasty and stent placement.

Zinc is a trace element involved in major regulatory processes and serves as a cofactor for a number of central enzymes. Zinc has been found at times to act as an anti-migratory agent, an anti-inflammatory agent, and a reducer of oxidative stress.

U.S. Pat. No. 6,264,595 discloses a radioactive transition metal stent which may be used to alleviate neointimal hyperplasia. The transition metal stent surface is chemically bound to a radioactive material without affecting the metallurgical properties of the transition metal. The transition metal may be any of 36 different metals, including zinc.

Visona, A. et al. (2000) J. Photochemistry and Photobiology 57:94-101 describes local delivery of photosensitizing agents followed by intraluminal local irradiation to prevent intimal hyperplasia and restenosis after angioplasty. The photosensitizing agent used was Zn(II)-phthalocyanine.

Khachigian L. M. (2001) Ann. N.Y. Acad. Sci. 947:412-5 discusses recent strategies for inhibiting post-angioplasty restenosis by targeting the zinc finger transcription factor and immediate-early gene product Egr-1.

### SUMMARY OF THE INVENTION

It is an object of the present invention to provide a method for preventing restenosis.

It is a further object of the invention to provide a device for use in preventing restenosis.

In one aspect of the invention, there is provided a method of reducing neointiminal proliferation in a blood vessel comprising administrating zinc to said blood vessel.

The present invention provides a novel method for reducing neointiminal proliferation in a blood vessel by using zinc. The term “reducing” is meant to include both preventing neointiminal proliferation as well as treating it by reducing the cell proliferation. However, the invention primarily relates to prevention.

The zinc is preferably free zinc, that is, zinc which is not conjugated or complexed e.g. with a protein or photosensitizer, such as a zinc cofactor. The term free zinc however includes various salts of zinc such as ZnCl₂, as well as zinc bound to or coated on various metals and other materials found in stents. The zinc may be administered either locally at the site of the neointimal proliferation, or systemically.

In the present specification, the term neointimal proliferation relates to the known pathological condition of blood vessels as occurs in particular, but not exclusively, in restenosis. As is well known, other pathological conditions such as high pulmonary blood pressure and cardiac transplantation can also bring on neointimal proliferation.

Local administration may be, for example, by use of a stent comprising zinc, as described below, or by a drug eluting device adapted to be inserted in a blood vessel such as an implanted mini-pump, or by other known modes of administration such as medial and adventitial infiltration, ‘sweating’ balloon, etc.

The local administration of the zinc may be carried out either alone or in conjunction with the administration of one or more anti-restenosis drugs, such as rapamycin, taxol, troglitazone, tranilast, dexamatazone, etc. It is expected that the use of zinc will allow a reduction in the therapeutic dosage of these drugs.

Systemic administration may be either oral by dietary supplementation or medication, or by injection such as i.v., i.m., i.p., etc. An example of a protocol may be daily i.p injections of 5 mg/Kg weight ZnCl₂ in normal saline. The amount of zinc administered will generally be at least 1 mg per administration. Preferably, the treatment should be started prior to the angioplasty procedure and continued for at least one month post-procedure.

A pharmaceutical composition comprising zinc and a pharmaceutically acceptable excipient is also contemplated by the invention.

In a second aspect of the invention, there is provided a stent comprising zinc, wherein the zinc is not chemically bound to a radioactive material.

The stent of the invention may be a conventional stent coated by zinc. Coating may be e.g. electromechemical or matrix coating.

The metal stent may be, for example, coated with a matrix allowing controlled release of zinc, or the zinc may be incorporated into a polymer-metal composite stent.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a graph illustrating the magnitude of the neointimal area of arteries taken from treated and control rats; and

FIG. 2 is a graph illustrating the magnitude of the neointimal/medial ratio of the arteries of FIG. 1.

### DETAILED DESCRIPTION OF THE INVENTION

In the following, an example is given to demonstrate the efficacy of zinc in controlling restenosis.

Carotid injury was induced in Wistar rats by balloon dilation according to a conventional model (Clowes A W, Reidy M A, Clowes M M. Kinetic of cellular proliferation after arterial injury. Lab Invest 1983; 49:327-334). Rats were then injected daily i.p. with either zinc (at a concentration of 5 mg/kg) or saline (control) for 2 weeks until sacrifice. Arteries were taken for morphometeric and immunohistochemical analysis.

Levels of serum zinc were elevated in the zinc-treated rats (248.2±19.4 pg/ml) as compared to the control rats (136.8±7.2 pg/ml; p<0.01).

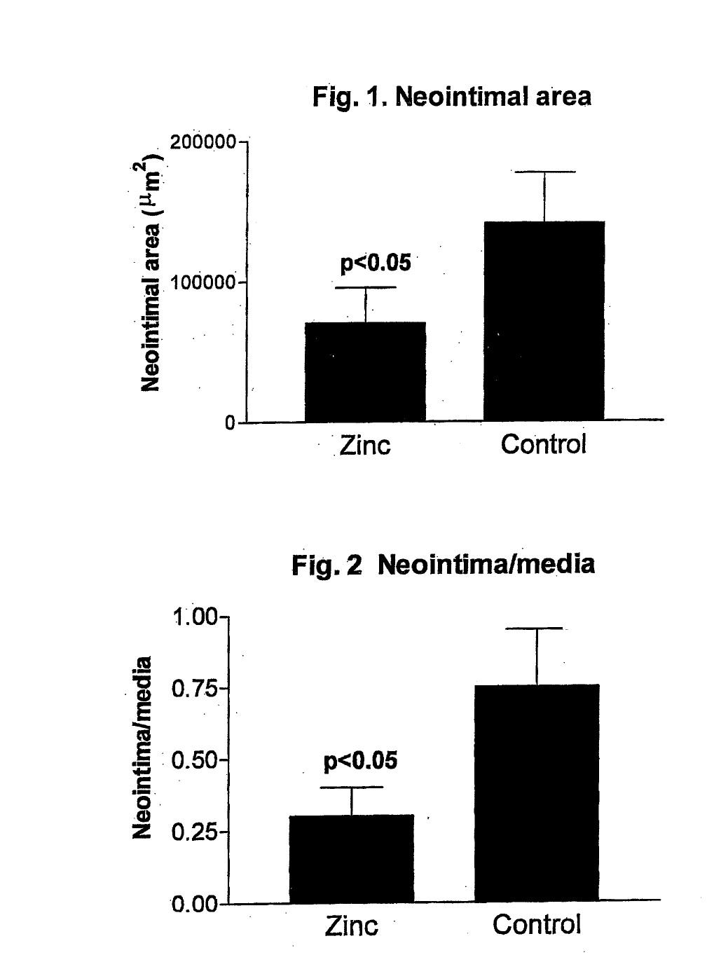

Zinc treatment reduced both neointimal area as well as the neointimal/medial ratio (FIG. 1 and FIG. 2, respectively) without influencing external elastic lamina size. By immunohisotochemical staining, a reduction was evident in inflammatory cell infiltrate (exhibited by NF-_{κ}B and iNOS positive cells) in the arteries obtained from the treated rats.

Thus Zinc was found to influence predominantly neointimal thickening and not negative remodeling.

1. A method of reducing neointiminal proliferation in a blood vessel comprising administrating zinc to said blood vessel.

2. A method according to claim 1 wherein the zinc is administered locally

3. A method according to claim 2 wherein the zinc is administered by use of a zinc-coated stent, wherein said stent does not contain radioactive material.

4. A method according to claim 3 wherein the stent is administered together with at least one anti-restenosis drug.

5. A method according to claim 2 wherein the zinc is administered by a drug eluting device adapted to be inserted in a blood vessel.

6. A method according to claim 1 wherein the zinc is administered systemically.

7. A method according to claim 6 wherein the zinc is administered by dietary supplementation.

8. A method according to claim 6 wherein the zinc is administered by injection.

9. A stent comprising zinc, wherein the zinc is not chemically bound to a radioactive material.

10. A stent according to claim 9 coated by zinc.

11. A drug eluting device adapted to be inserted in a blood vessel comprising zinc.

12. A pharmaceutical composition for use in reducing neointiminal proliferation comprising zinc and a pharmaceutically acceptable excipient.

13. A composition according to claim 12 in a form adapted for oral administration.

14. A composition according to claim 12 in a form adapted for administration by injection.
